(19) United States
(12) Patent Application Publication
(10) Pub. No.: US 2004/0002444 A1
(43) Pub. Date: Jan. 1, 2004

(54) Polyphosphate-water soluble collagen complexes and process for preparation thereof

(75) Inventors: Toshikazu Shiba, Tokyo (JP); Toshie Shiba, Nagano (JP); Minoru Yamaoka, Nagano (JP); Takashi Uematsu, Nagano (JP); Yoshiharu Takahashi, Nagano (JP); Hitoshi Tanaka, Nagano (JP)

Correspondence Address: SUGHRUE MION, PLLC, 2100 Pennsylvania Avenue, NW, Washington, DC, 20037-3213, US

(73) Assignees: Toshikazu SHIBA; SHIBARIE RESEARCH INSTITUTE

(21) Appl. No.: 10/408,778

(22) Filed: Apr. 8, 2003

(30) Foreign Application Priority Data
Apr. 8, 2002 (JP) 2002-105468

(51) Int. Cl. A61K 38/16

(52) U.S. Cl. 514/7

(57) ABSTRACT

A polyphosphate-collagen complex, comprising at least one polyphosphate bound to collagen, a process for preparing the complex, and a method for promoting tissue regeneration using the complex.

This application claims the benefit of a priority of an earlier Japanese patent application No. 2002-105468 filed on Apr. 8, 2002, which is incorporated herein, by reference, in its entirety.

### BACKGROUND OF THE INVENTION

1. Field of the Invention

The present invention relates to a biocompatible material, which promotes the tissue regeneration of various biological tissues such as bones, skin, and internal organs, and a process for preparing the same.

2. Related Art

Polyphosphate has been reported to have a cell growth effect and a tissue regeneration effect by stabilizing cell growth factors such as FGF (JP Patent Publication (Kokai) No. 2000-069961). It has been reported that the polyphosphate also has an effect of promoting bone differentiation and thus has an effect on bone regeneration (JP Patent Publication (Kokai) No. 2000-079161). Further, the safety of the polyphosphate for the living body has long been confirmed, and it is known to be a biodegradable substance that degrades in vivo into atoxic phosphoric acids.

On the other hand, polyphosphates are normally provided as an aqueous solution. To use a polyphosphate's tissue regeneration effect on biological tissues, it becomes necessary to mix the polyphosphate with a base material that allows the polyphosphate to reside at a lesion for a certain time to maintain the effect. Accordingly, it has been conventionally attempted to prepare a composite material by impregnating a biocompatible material (base material) such as a collagen sponge, collagen sheet, carboxymethyl cellulose or polylactic acid with a polyphosphate aqueous solution. The composite material is then used as a polyphosphate-containing material for tissue regeneration.

However, the method, which involves impregnating a base material with a polyphosphate aqueous solution, has a difficulty allowing the base materials to uniformly contain a precise amount of polyphosphate. This makes it difficult to prepare polyphosphate-containing products having a uniform quality. Further, the polyphosphate possesses a characteristic of not adhering firmly to a base material itself. Hence, there are other problems in that when applied to a lesion, the polyphosphate alone is easily freed from the base material, and it is degraded. Furthermore, the base material itself is deformed by the process of impregnation with polyphosphate, so that it can become difficult to use the base material in a form that is easily applicable to a lesion. On the contrary, it is also difficult to form a base material that is easily applicable to a lesion after the impregnation of the base material with polyphosphates.

### SUMMARY OF THE INVENTION

To address the above problems raised under conditions wherein polyphosphates and base materials are mixed, an object of the present invention is to provide a material containing polyphosphates and base materials, which is more easily applicable as a medical material, and can allow the polyphosphate to be able to effectively exert action to promote tissue regeneration.

As a result of considerable effort to achieve the above objective, we have completed the present invention by finding that an insoluble complex, wherein a polyphosphate is bound to collagen, can be prepared by mixing polyphosphoric acids and collagens under specific conditions.

That is, the present invention is as follows.

[1] A polyphosphate-collagen complex, comprising at least one polyphosphate bound to collagen.

In this complex, the polyphosphate can be at least one type represented by a general formula: (PₙO₃ₙ₊₁)⁽ⁿ⁺²⁾⁻ (wherein “n” indicates an integer between 2 and 5000). This polyphosphate can also be a linear condensed polyphosphate. The polyphosphate is preferably represented by the above formula wherein “n” is an integer between 20 and 2000. Further, the above collagen is preferably an atelocollagen. Furthermore in the above complex, the weight ratio of polyphosphate to collagen is preferably between 0.1% :99.9% and 20% :80%.

[2] A medical material for promoting tissue regeneration, containing the complex of [1] above.

[3] A medical material for treating periodontal disease, containing the complex of [1] above.

[4] A process for preparing a polyphosphate-collagen complex, which comprises mixing a polyphosphate solution at a concentration of between 0.5 and 10% by weight with a collagen solution at a concentration of 0.1 to 10% by weight, and collecting the thus generated precipitate.

The step of mixing in this process is preferably performed under pH conditions between 5.0 and 8.0. Further, the polyphosphate solution used in the process can be a solution of at least one polyphosphoric acid represented by the general formula: (PₙO₃ₙ₊₁)ⁿ⁺²⁾ H (wherein “n” indicates an integer between 2 and 5000) or a salt thereof. The polyphosphoric acid can be the one represented by the above general formula wherein “n” is preferably an integer between 20 and 2000. The polyphosphate solution may be a solution of a polyphosphoric acid having the average chain length of 60 to 70 or a salt thereof. Further, collagen contained in the collagen solution is water-soluble collagen, and particularly preferably atelocollagen. Further, the preferred weight ratio of polyphosphate to collagen in the prepared polyphosphate-collagen complex is between 0.1% :99.9% and 20% :80%.

[5] A method for concentrating and separating polyphosphates having an average chain length of 60 to 70, which comprises mixing, at a volume ratio of between 2:1 and 9:1, a hexametaphosphate solution at a concentration of between 0.1 and 10% by weight with 87 to 100% ethanol, and separating the precipitated polyphosphates from a reaction solution. Here, “average chain length” means the average value of the above “n.”

[6] A method for promoting tissue regeneration, which comprises applying the complex of [1] above to the tissue, for which tissue regeneration should be promoted. This method is particularly preferable for a case wherein a subject tissue is periodontal tissue, bone, or burned or wounded tissue. The complex can be applied to such a tissue by, for example, coating, embedding, spraying or injecting the complex into the tissue, or covering tissue with the complex. However, the application method is not limited to these methods.

### BRIEF DESCRIPTION OF THE DRAWINGS

FIG. 1 shows the molecular weight distribution of polyphosphates obtained by the method for concentrating and separating medium chain polyphosphates of the present invention as analyzed by polyacrylamide gel electrophoresis. Lanes 1 to 5 denote chain length markers for polyphosphates. Lane 1: average chain length of 5, Lane 2: average chain length of 15, Lane 3: average chain length of 35, Lane 4: average chain length of 45, Lane 5: average chain length of 65, Lane 6: sodium hexametaphosphate before the separation of medium chain polyphosphates by solvent separation (average chain length of 15), and Lane 7: medium chain polyphosphates separated by solvent separation (average chain length of 60 or more).

FIG. 2 includes photographs of stained tissues showing the regenerated alveolar bone of rats that were treated with the polyphosphate-collagen complex (treated group: FIG. 2B) or with collagen (comparison group: FIG. 2A).

FIG. 3 is a graph showing changes in the area of the regenerated alveolar bone of rats that were treated with the polyphosphate-collagen complex (treated group) or with collagen (comparison group). “●” denotes the treated group that was treated with the polyphosphate-collagen complex, and “◯” denotes the comparison group (control group) that was treated only with collagen. The symbol “*” shows that the polyphosphate-collagen complex's effect of promoting the regeneration of the alveolar bone of the treated group that was treated with the complex is significant with P<0.05 compared with the comparison group in the same week.

FIG. 4 is a graph showing the areas of the regenerated alveolar bone of the rats that were treated with polyphosphate-collagen complexes that had been prepared using polyphosphates with different average chain lengths. Treated group 1 denotes a group that was treated with a polyphosphate-collagen complex containing polyphosphates with an average chain length of 15, treated group 2 denotes a group that was treated with a polyphosphate-collagen complex containing polyphosphates with an average chain length of 35, treated group 3 denotes a group that was treated with a polyphosphate-collagen complex containing polyphosphates with an average chain length of 75, comparison group 1 denotes a group that was treated only with collagen, and comparison group 2 denotes a group that was treated with a mixed solution of a phosphate buffer solution and collagen. The symbol “*” shows that the effect of promoting the regeneration of the alveolar bone is significantly high with P<0.01 in the groups, compared to the effect in comparison group 1.

### DETAILED DESCRIPTION OF THE PREFERRED EMBODIMENTS

The present invention relates to a complex, wherein polyphosphates having an action to promote tissue regeneration are bound to a base material. When the complex is separated and used as a material, it can be a medical material that can allow the polyphosphates to exert the tissue regeneration promotion action in biological tissues.

In the present invention, to prepare a complex of polyphosphates and the base material, collagen is selected as the most preferable base material. According to the present invention, insoluble polyphosphate-collagen complexes can be formed by mixing a polyphosphate solution with a collagen solution under certain conditions that are described later. This complex is insoluble, takes a gel form, and can be easily separated by filtration or centrifugation from a solution. The gel polyphosphate-collagen complex can be applied intact as a material for promoting tissue regeneration to a lesion, or can also be dried, and then easily formulated as a block, sponge, sheet, fiber, mesh or the like. Further, the complexes are useful, because they have uniform polyphosphate contents, and polyphosphates are not easily freed from a base material, so that the complex can be produced as a medical material provided with uniform quality. The medical material of the present invention may be the polyphosphate-collagen complex according to the present invention, to which other components (a crosslinker, a biologically active substance, and the like) are added, if necessary. In addition, the form of the medical material of the present invention is not particularly limited. It may exist in a gel form, or may be in a desired form that is formulated after drying.

The present invention is explained in detail as follows.

1. Polyphosphate

Examples of a polyphosphate that is bound to collagen to form the complex of the present invention include, but are not limited to, a linear condensed polyphosphate (including an hexametaphosphate) that is obtained by dehydration and condensation of orthophosphoric acids, a side chain polyphosphate that has organic groups introduced at the side chain, and a cyclic polyphosphate. Preferably, the polyphosphates in the complex are polyphosphate ions represented by the general formula “(PₙO₃ₙ₊₁)ⁿ⁺²⁾⁻” and are, in particular, linear condensed polyphosphate ions having a structure wherein two or more phosphate (PO₄) tetrahedrons are linked linearly by sharing oxygen atoms at the apexes. In the present invention, “n” in the above general formula is an integer that is at least 2, preferably between 2 and 5000, more preferably between 5 and 5000, further preferably between 15 and 2000, and most preferably between 20 and 2000. In the present invention, when a medium chain polyphosphate is used, “n” in the above general formula can be preferably between 20 and 1000, more preferably between 30 and 500, further preferably between 50 and 200, and still further preferably between 60 and 100. In this specification, the chain length of a polyphosphate means the number of polymerized phosphoric acids (the value of “n” in the above general formula). Thus, “average chain length” represents the mean of the numbers of polymerized phosphoric acids (“n” in the above general formula).

In preparation of the polyphosphate-collagen complex of the present invention, as a source of the above polyphosphate ion, for example, a polyphosphoric acid such as a linear condensed polyphosphoric acid (including an hexametaphosphate) that is obtained by dehydration and condensation of an orthophosphoric acid, a side chain polyphosphoric acid that has organic groups introduced at the side chain, or a cyclic polyphosphoric acid, and/or a solution of a salt thereof can be used. Preferably, such a polyphosphoric acid may be a compound represented by a general formula: (PₙO₃ₙ₊₁)⁽ⁿ⁺²⁾ H, wherein “n” is an integer that is at least 2, preferably between 2 and 5000, more preferably between 5 and 5000, further preferably between 15 and 2000, and most preferably between 20 and 2000. When a medium chain polyphosphoric acid is used, “n” in the above general formula is preferably between 20 and 1000, more preferably between 30 and 500, further preferably between 50 and 200, and still further preferably between 60 and 100. More generally, in a polyphosphoric acid that is used in preparation of the polyphosphate-collagen complex of the present invention, the number of polymerized phosphoric acids is between 2 and 5000, preferably between 5 and 5000, more preferably between 15 and 2000, and further preferably between 20 and 2000. In the present invention, when a medium chain polyphosphoric acid is used, the number of polymerized phosphoric acids in the polyphosphoric acid is preferably between 20 and 1000, more preferably between 30 and 500, further preferably between 50 and 200, and still further preferably between 60 and 100. In the present invention, “a salt of a polyphosphoric acid” is a compound having a molecular structure wherein hydrogen of a hydroxyl group of the polyphosphoric acid is substituted with a metal. Examples of the metal in this case include sodium, potassium, calcium and magnesium.

The polyphosphate solution that is used in preparation of the above complex may contain one type or multiple types of the above-described polyphosphoric acids or salts thereof. Examples of the multiple types of polyphosphoric acids or salts thereof include polyphosphoric acids having different polymerization degrees or salts thereof, polyphosphoric acids having different molecular structures or salts thereof, and polyphosphate salts having different metal ions. In addition, the polyphosphate solution may contain both polyphosphoric acids and salts thereof. Moreover, when such a solution containing multiple types of polyphosphoric acids or salts thereof is used in preparation of the above complex, the polyphosphates contained in the thus prepared polyphosphate-collagen complex can be multiple types of polyphosphates. Accordingly, the polyphosphates contained in the polyphosphate-collagen complex of the present invention can be one type of or multiple types of polyphosphates.

In terms of the tissue regeneration promotion action of a polyphosphate, a polyphosphate having a chain length of 20 or more has a greater effect than that having a short chain length of less than 20. For example, in an experiment to examine the promotion of bone differentiation of osteoblasts that is an indicator of bone regeneration by adding polyphosphates to a culture of osteoblasts, the use of a polyphosphate having an average chain length of 25 or 35 leads to a higher effect of promoting bone formation than the use of a polyphosphate having an average chain length of 15 (n=15). Accordingly, to allow polyphosphates to exert the action of promoting tissue regeneration by the use of the complex of the present invention, it is more advantageous to use a polyphosphate having a chain length of 20 or more. However, a generally commercially available low-cost polyphosphate is a salt of hexametaphosphate salt (such as, sodium hexametaphosphate, potassium hexametaphosphate, and the like), which is a linear polyphosphate having an average chain length of approximately 15 for industrial use or for food additives. Most polyphosphates contained in the hexametaphosphate salt have a chain length of less than 20, and the content (percentage) of polyphosphates having a chain length of 20 or more is very low. Hence, in the present invention, a novel method for obtaining a medium chain polyphosphate having a chain length of 20 or more using such a low-cost, easily available hexametaphosphate salt has been developed.

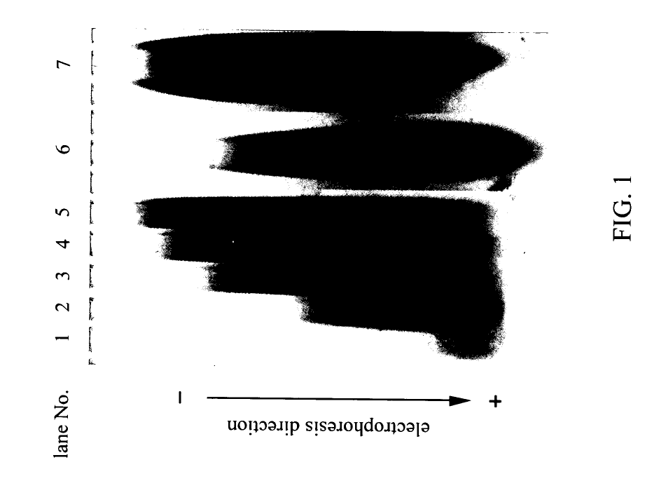

In this method, first, hexametaphosphate salts are dissolved in water to have 0.1 to 10% by weight, or preferably 10% by weight. To the hexametaphosphate aqueous solution, 87% to 100% ethanol, preferably 96% ethanol is added with 1/10 to ⅓ the volume of the entire liquid volume after mixing of the hexametaphosphate solution with the ethanol. That is, the ethanol is added in a volume, by which the volume ratio of hexametaphosphate aqueous solution to ethanol is between 2:1 and 9:1. The mixed solution is agitated sufficiently. The thus obtained precipitate is separated from the aqueous solution fraction by a separation method, such as, but are not limited to, centrifugation or a filter filtration. The thus separated precipitate is a medium chain polyphosphate. Subsequently, the polyphosphate is washed with 70% ethanol, and then dried. The average chain length of polyphosphates obtained by the above-described separation procedure ranges from 60 to 70, and the product merely contains short chain polyphosphates having a chain length of 10 or less (see lanes 1, 2, and 7 in FIG. 1). Therefore, the polyphosphates obtained by this method have higher tissue regeneration promotion effect. As described above, the method for concentrating and separating polyphosphates of the present invention is useful, because, with this method using very low-cost commercial available hexametaphosphate salts, medium chain polyphosphates having a high effect to promote tissue regeneration can be efficiently separated and concentrated.

A medium chain polyphosphate that is obtained as described above, particularly a polyphosphate having an average chain length of 60 to 70, can be preferably used in preparation of the polyphosphate-collagen complex of the present invention.

2. Collagen

As a base material for forming the complex of the present invention, collagen is used as a material having biocompatibility. Collagen is a fibrous protein that has a striated structure. Examples of such collagen include various collagens such as, but are not limited to, collagen type I, type III and type V that are mainly obtained from tissues other than the cartilage, collagen type II that is mainly obtained from the cartilage tissue, and collagen type IV and type VI that form network-like aggregates. In the present invention, these collagens are preferably used as water-soluble collagens. Hence, in the present invention, these collagens are preferably atelocollagens, in which telopeptides at both ends of the protein fiber have been removed. As the collagen of the present invention, any water-soluble collagen derived from the tissue of animals including, for example, cattle, pigs, chickens, and fish such as salmon can be used. However, the collagens used in the present invention are not limited to those derived from nature. The collagen may be obtained from the biological tissue of an animal, or may be obtained artificially by protein production in microorganisms or culture cells using genetic engineering techniques. When applied into a human body, with consideration given to antigenicity and risk of infection with microorganisms, a human-derived collagen or a genetic recombinant human collagen is preferably used. Further, the collagen used in the present invention may be a derivative, as long as it does not obstruct the formation of the polyphosphate-collagen complex as described later. An example of the collagen derivative is one wherein various functional groups such as a hydroxyl group, a carboxyl group, an amino group, a cyano group, a thiol group, a saturation or unsaturation alkyl group, a benzene ring or a group having a heterocyclic group are introduced by a chemical bond such as ester, ether, amide, urethane, or urea bond.

3. Preparation of Polyphosphate-Collagen Complex

A process for preparing the complex of the present invention using the above polyphospahte and collagen is as described below.

The above-described polyphosphoric acid or the salt thereof used as a supply source of polyphosphate ions for the complex are used in the form of a polyphosphate solution. The polyphosphate solution may be dissolved in sterile distilled water, or in a buffer solution, such as Tris-HCl buffer or phosphate buffer. At this time, the concentration of polyphosphates is between 0.5 and 10% by weight, and preferably between 1 and 5% by weight. The polyphosphate solution with such a concentration is mixed with a 0.1 to 10% by weight, or preferably 0.1 to 2% by weight collagen solutions. Herein, the concentrations of the polyphosphate solution and collagen solution are shown with percentage of the dry weight (of each polyphosphoric acid or a salt thereof, or collagen dissolved therein) to the total weight of each of these solutions. When mixing is performed, pH preferably ranges from 5.0 to 8.0, and particularly preferably from 7.0 to 8.0. When polyphosphates and collagens are mixed under such conditions, opaque gel substances are precipitated. The precipitate is the polyphosphate-collagen complex.

This complex can be separated from the aqueous solution fraction by any separation method, such as centrifugation or mesh filtration, and then washed several times with sterile distilled water. After washing, the gel substances can be used intact as a polyphosphate-collagen complex material. Alternatively, the polyphosphate-collagen complex can be used and stored after drying. In this case, for example, the complexes are put in various forms by means of molds, and vacuum freeze-dried, so that desired forms of the polyphosphate-collagen complex materials can be prepared. In addition, the precipitation of the polyphosphate-collagen complex is a phenomenon that can occur limitedly under the herein described conditions of the solution concentration range and pH. There has been no report concerning the formation of polyphosphate-collagen complexes by the process described in this specification, or the complex itself.

The polyphosphate-collagen complex obtained by the above method has a weight ratio (after drying) of polyphosphate to collagen of between 0.1%: 99.9% and 20% :80%, and preferably of 4.1% :95.9%. The weight ratio after drying can be measured and calculated by conventionally known techniques. For example, the phosphorus atom and nitrogen atom contents in the dried complex can be measured by plasma emission spectral analysis. Then, from the phosphorus atom and nitrogen atom contents in the complex, the polyphosphates and collagen (protein) contents can be calculated. Alternatively, the dry weight of polyphosphates, which participate in the formation of a complex, can also be calculated by subtracting the dry weight of only the collagen used in the formation of the complex from the dry weight of the formed polyphosphate-collagen complex. Percentages of the thus calculated weight of polyphosphates, which participate in the formation of the complex, and the weight of only the collagen used for the synthesis of the complex, are calculated to the weight of the generated polyphosphate-collagen complex. Then, these values of the percentages are represented in the form of a ratio, so that the weight ratio of polyphosphate to collagen in the polyphosphate-collagen complex can be determined. The thus measured weight ratio after drying is obtained as a uniform value for the obtained polyphosphate-collagen complex of the present invention, which is prepared under conditions within a certain range. That is, the polyphosphate-collagen complex of the present invention may be a uniform substance wherein a certain amount of polyphosphates is bound to collagens.

4. Medical Material for Promoting Tissue Regeneration Containing a Polyphosphate-Collagen Complex

The polyphosphate-collagen complex prepared by the above method can be used as a biocompatible material for various applications. In particular, the polyphosphate-collagen complex of the present invention can be used for various medical materials for promoting tissue regeneration using the tissue regeneration promotion action of polyphosphate.

When the polyphosphate-collagen complex of the present invention is used for various materials, any additives or the like can be further added appropriately to the complex in a gel form. For example, the flexibility of the resulting material can be altered by adding a crosslinking agent to the complex. In this case, for example, a polyphenol crosslinking agent is added, so that the collagens in the complex can be cross-linked. As the polyphenol crosslinking agent, in terms of biocompatibility, bio-related substances such as tannic acid and lignin are appropriate. The amount of such a crosslinking agent to be used herein is preferably between 0.05% and 5% of the amount of collagen so as not to inhibit collagen gelation due to cross-linking. Further, polyphosphates in the complex can be cross-linked by irradiation of the above complex with ultraviolet rays. Moreover, any cross-linking method known by a person skilled in the art can be used for the complex of the present invention. Such a method can involve allowing an appropriate compound to covalently bind to the terminus or anywhere between the ends on the chain of a polyphosphate, and cross-linking the polyphosphates through the compound.

As an additive, a substance having biological activity may be further added to the complex of the present invention. Examples of such an additive include ascorbic acid that activates collagen synthesis; a biocompatible artificial bone component such as calcium phosphate, and hydroxyapatite; an antibiotic such as penicillin, cefem, and tetracycline-based pharmaceutical preparations; a transforming growth factor such as the TGF-β superfamily; and a bone morphogenetic protein such as BMP-1, BMP-2 and BMP-3.

The polyphosphate-collagen complex of the present invention appropriately prepared as a material as described above can be advantageously used as a medical material for promoting tissue regeneration, such as a medical material for treating periodontal disease, a medical material for promoting bone regeneration and a material for facilitating preparation of artificial organs.

Particularly in the regeneration of the periodontal tissue, for example, approximately 0.05 to 0.5 ml of the above complex in a gel form is injected into the periodontal pocket, so as to allow the complex to exert the function of aggressively repairing tissues that have been damaged by periodontal disease, such as damaged alveolar bone, periodontal ligament, and dental cement. Further, when the above complex is dried and formulated into a sheet shape and embedded in the periodontal pocket after the removal of a site contaminated by bacteria, the complex can be very efficiently used to promote tissue regeneration in ENAP surgical operation, flap operation, or GTR method. Such a sheet-shaped complex may be used by formulating it into, for example, the one with a thickness of between 0.1 and 3 mm, and between 0.25 and 100 cm. It is appropriately cut and deformed according to the shape of a lesion, that is, the periodontal pocket. Further, the complex can be formulated into a fibrous form, and then complexes can be prepared as cloth or mesh materials using the fiber. The thus prepared complex material can be used after appropriate processing such as cutting and deforming the material according to the area and shape of a lesion, similarly to the sheet-shaped complex material.

The complex of the present invention can be appropriately used not only as a material for promoting periodontal tissue regeneration, but also for regeneration of other tissues. To promote bone regeneration, the gel complex of the present invention is applied intact to the gap between the broken bone ends, so that promotion of bone regeneration can be expected. The amount of the complex to be applied can be appropriately determined according to the affected area of a bone fracture site. Moreover, the complex is formulated into a block shape or a shape suitable for the lost portion of bone at a lesion, and then the formulated complex can be directly used as a material for promoting the repair of the lost portion of the bone. For example, in a case wherein a part of a bone is lost as a result of treatment for a disease such as osteosarcoma, the complex can be used by applying it to the lost portion of the bone, in order to fill the lost portion and promote bone regeneration. Such a material for promoting the repair of the lost portion of a bone can be formulated into, for example, 0.1 to 100 g in a cylindrical shape.

Further, the complex of the present invention can also be used as a material for artificial skin, artificial organs or the like to promote tissue regeneration. In the case of artificial skin, the complex material processed into a sheet or mesh material is applied to, for example, a burn lesion, to promote the growth of the cells of the corium and epidermis, so as to be able to contribute to the early cure of the wound. In this case, the complex as a sheet or mesh material may be used by cutting the material into an area and shape so that it covers the entire lesion. When a wound is an incised wound or the like, the gel complex is directly applied to the lesion so as to be able to promote tissue regeneration. The dose in this case can be appropriately determined according to the area of a lesion, and is preferably between 0.1 and 1 g per 10 cm² of a lesion.

### EXAMPLE

The present invention will be described more specifically by examples, but the technical scope of the present invention is not limited by these examples.

### Example 1

### Concentration and Separation of Medium Chain Polyphosphate

20 g of sodium hexametaphosphate as specified in the standard for food additives was dissolved in 200 ml of purified water, and then 32 ml of 96% ethanol was gradually added to the solution. The solution was agitated well, and then allowed to stand at room temperature for approximately 30 minutes. Then, centrifugation (10,000×g, 20 minutes, 25° C.) was performed, so as to separate precipitate from the aqueous solution fraction. The aqueous solution fraction was discarded. 70% ethanol was added to the collected precipitate for washing, and then vacuum-dried. Thus, 9.2 g of medium chain polyphosphate salts was obtained as a precipitate (yield 46.0%). Further, the polyphosphate salt was subjected to polyacrylamide gel electrophoresis, and then the molecular weight was analyzed. 15% polyacrylamide gel was used for electrophoresis. After electrophoresis, the gel was stained using toluidine blue, and then observed. The approximate average chain length (or average molecular weight) of the polyphosphate contained in the sample was estimated by simultaneously performing electrophoresis for polyphosphates having known chain lengths as chain length markers, and then by comparing the results with the results of this electrophoresis. The molecular weight distribution, which shows the results of this analysis, is shown in FIG. 1. Lanes 1 to 5 denote chain length markers. Lane 1 indicates a polyphosphate with an average chain length of 5, lane 2 an average chain length of 15, lane 3 an average chain length of 35, lane 4 an average chain length of 45, and lane 5 an average chain length of 65. As tested samples, a sodium hexametaphosphate was loaded on lane 6 before the separation of medium chain polyphosphates due to solvent separation, and the medium chain polyphosphates separated by solvent separation were loaded on lane 7.

As shown in FIG. 1, the polyphosphates extracted from sodium hexametaphosphates as specified in the standard for food additives (before separation; approximate average chain length of 15, FIG. 1, lane 6) by solvent separation using alcohol showed a migration degree (after separation; FIG. 1, lane 7) that was almost equivalent to that of the polyphosphate as the chain length marker having an average chain length of 65 shown in lane 5. Thus, it was suggested that medium chain polyphosphates having an average chain length of 60 or more were contained. Therefore, it was shown that medium chain polyphosphate salts having an average chain length of 60 or more can be extracted and separated from hexametaphosphate salts by the method of the present invention for concentrating and separating medium chain polyphosphates.

### Example 2

### Preparation of Polyphosphate-Collagen Complex

10.0 g of the medium chain polyphosphate salts that had been separated from sodium hexametaphosphate by the method described in Example 1 was dissolved in 1000 ml of sterile distilled water. 286 g of a chicken-derived atelocollagen (Atelo Helogen) (1.72 g of solid content) was added at room temperature, thereby generating a gel precipitate. The precipitate was filtered with mesh, and then washed with 70% ethyl alcohol, so that complexes with a wet weight of 129.3 g were obtained. The complexes were further dried using a vacuum dryer, so that dried polyphosphate-collagen complexes with a dry weight of 2.74 g could be obtained.

The content of phosphorus atom contained in the thus prepared polyphosphate-collagen complex was quantitatively analyzed by the plasma emission spectral analysis method. Thus, phosphorus atoms contained in the complex represented approximately 4.5% thereof. Since the amount of phosphorus atoms contained in the collagen that had been used for preparing the complex was below detection limit, it was demonstrated that polyphosphates were bound to collagens to form the complexes. Further, the phosphorus atom content in the polyphosphate salts employed herein was 29.8%, as similarly measured by the plasma emission spectral analysis method. The obtained result was almost equivalent to the theoretical value, 29.5%. Further, when the concentration of polyphosphate to be used upon the preparation of the complex was varied, the phosphorus atom content contained in the resulting complex, specifically, the content of polyphosphate in the complex, did not change. Hence, it was shown that the polyphosphate-collagen complex of the present invention comprised a certain amount of polyphosphates bound to collagens. Furthermore, the dry weight ratio of polyphosphate to collagen in the polyphosphate-collagen complex obtained in this example was found by the following calculation method. Specifically, the dry weight of polyphosphates, which participated in the formation of the complex, was calculated by subtracting the dry weight of only the collagen used in the synthesis of the complex from the dry weight of the formed polyphosphate-collagen complex. To the dry weight of the polyphosphate-collagen complex, percentages of the thus calculated dry weight of the polyphosphates, and of the dry weight of only the collagen used for the synthesis of the complex were calculated. Then, these values were represented by the form of a ratio, so that the weight ratio of polyphosphate to collagen in the complex was determined. As a result, the weight ratio of polyphosphate to collagen in the polyphosphate-collagen complex prepared in this example was 4.1% :95.9%.

### Example 3

### Effect of Polyphosphate-Collagen Complex in Promoting Tissue regeneration

Next, to confirm the effect of the prepared polyphosphate-collagen complex in regenerating the periodontal tissue, an experiment for the regeneration of the periodontal tissue was performed using rats. Wister male rats (8 weeks old; 18 rats in total) were anesthetized. Then, using a ½ round bar, approximately 2 mm portions were removed from the buccal alveolar bone crest of the mandibular first and second molars, and artificial periodontal pockets (gingival crevices) were formed. In the treated group (9 rats), approximately 0.1 ml of the polyphosphate-collagen complexes prepared in Example 2 was injected using a syringe into the thus formed gingival crevices. In the comparison group (9 rats), only the collagen was injected. The injection procedure was performed everyday from the day following the operation to prepare the gingival crevice, and continued for 3 weeks at the longest.

The rats treated for a certain period were euthanized by inhalation anesthesia (enflurane), and the tissues were fixed by perfusion fixation. Perfusion fixation was performed by letting 300 ml of physiological saline and then 10% neutral buffered formalin solution (pH 7.4, 500 ml) to flow through the blood vessel. After perfusion fixation, the mandible was cut, and then fixed by penetration of the above 10% neutral buffered formalin solution for 24 hours at 4° C. Later, decalcification was performed using 10% ethylene diamine tetraacetic acid solution at 4° C. for approximately 2 weeks. After decalcification, the samples were trimmed by cutting at the second molars. The trimmed samples were embedded with their cut surfaces down, and then frozen at −80° C. Tissue sections were prepared from the frozen samples, stained, and then observed under a light microscope. In addition, the area of the regenerated alveolar bone was measured as the area of bone formation by calculating the sum of the bone areas in the sections with image processing.

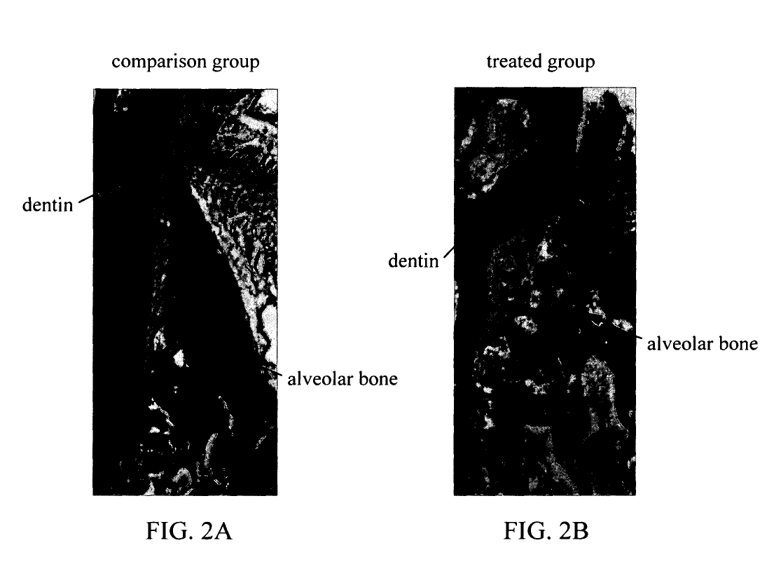

FIG. 2 shows the results of staining of the tissues of rats that were injected with the polyphosphate-collagen complex (treated group) or only with collagen (comparison group). In the treated group, significantly regenerated alveolar bones were observed, and the bone reconstruction was also significant. In the comparison group, however, only the cured gingival crevice was observed, and no increase was observed in the alveolar bone.

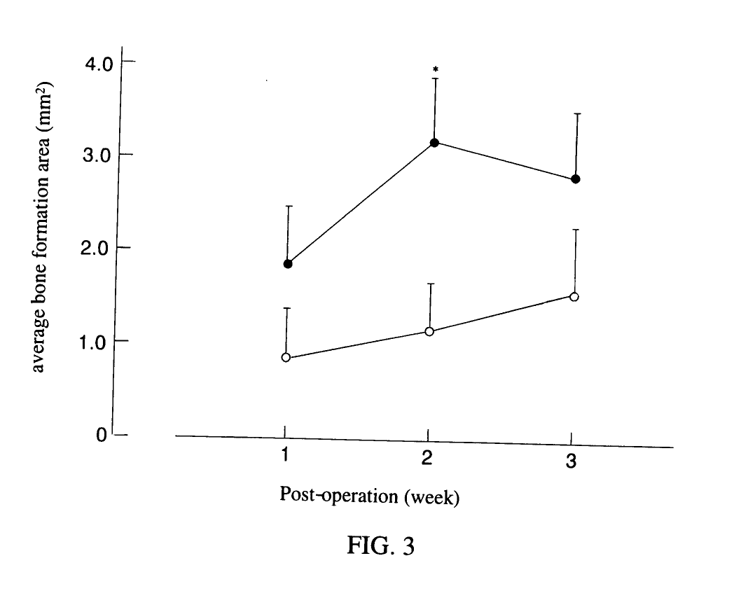

FIG. 3 shows changes in the area of bone formation. “●” denotes the treated group that was treated with the polyphosphate-collagen complex, and “◯” denotes the comparison group (control group) that was treated only with collagen. The symbol “*” indicates that the polyphosphate-collagen complex's effect of promoting the regeneration of the alveolar bone of the treated group that had been treated with the polyphosphate-collagen complex was significant with P<0.05, compared with the comparison group in the same week. In the polyphosphate-collagen complex-treated group (●), significant increases were shown in the area of the regenerated alveolar bone, compared with the comparison group (◯) that was treated only with collagen. In the polyphosphate-collagen complex-treated group, already at 1 week after the treatment, the area of the regenerated alveolar bone reached nearly double that of the comparison group, suggesting significantly promoted regeneration of the alveolar bone. As described above, the polyphosphate-collagen complex was shown to be able to exert significantly and earlier the outstanding effect of promoting bone regeneration.

### Example 4

### Influence of the Chain Length of Polyphosphate in the Effect of Promoting the Regeneration of Alveolar Bone

3 types of polyphosphate-collagen complexes were prepared using sodium polyphosphates having average chain lengths of 15, 35, and 75 (phosphate glass, Sigma). First, 1 g each of sodium polyphosphates respectively having each of the above average chain lengths was dissolved in 100 ml of sterile distilled water under pH conditions of 7.5, so that 3 types of polyphosphate solutions were prepared. Then, 28.6 g of a chicken-derived atelocollagen (Atelo Helogen) (0.172 g of solid content) was added to each solution at room temperature, thereby generating gel precipitates. These precipitates were filtered with mesh, and then washed with 70% ethyl alcohol, so that 3 types of polyphosphate-collagen complexes respectively containing polyphosphates with average chain lengths of 15, 35 and 75 were obtained.

Next, an experiment of the regeneration of the periodontal tissue in rats was performed using the 3 prepared types of polyphosphate-collagen complexes. In a manner similar to Example 3, Wister male rats (8 weeks old) were anesthetized. Then, using a ½ round bar, approximately 2 mm portions were removed from the buccal alveolar bone crest of the mandibular first and second molars, and artificial periodontal pockets (gingival crevices) were formed. In the treated group, approximately 0.1 ml of either one type of the above polyphosphate-collagen complexes was injected using a syringe into the thus formed gingival crevices. The treated groups were: treated group 1 that was treated with the polyphosphate-collagen complex containing polyphosphates having an average chain length of 15; treated group 2 that was treated with the polyphosphate-collagen complex containing polyphosphates having an average chain length of 35; and treated group 3 that was treated with the polyphosphate-collagen complex containing polyphosphates having an average chain length of 75. The comparison groups were: comparison group 1 that was treated only with collagen and comparison group 2 that was treated with a mixed solution of a sodium phosphate buffer and collagen. Injection (approximately 0.1 ml) into the gingival crevices of each group was performed similarly to that for the treated groups. For all the groups, 3 rats were used per group. In addition, the injection was performed once a day and everyday from the day following the operation to prepare the gingival crevice, and was continued for 14 days.

After the above treatment, at 15 days after the operation to prepare the gingival crevice, tissue sections (samples) were prepared in a manner similar to that in Example 3 from the rats of each group, and then the area of the formed alveolar bone was measured. The areas of the regenerated alveolar bones as measured for rat individuals included in 3 treated groups and 2 comparison groups are as shown in Table 1.

TABLE 1

| | Group treated with complex | | | | |
|---|---|---|---|---|---|
| | Treated | Treated | Treated | Comparison group | |
| | group 1 | group 2 | group 3 | | Comparison |
| | [average | [average | [average | Comparison | group 2 |
| | chain length | chain length | chain length | group 1 | [collagen + |
| | of 15] | of 35] | of 75] | [collagen] | Na phosphate] |
| | (mm²) | (mm²) | (mm²) | (mm²) | (mm²) |
| Individual 1 | 1.8772 | 2.7661 | 3.0112 | 0.5422 | 0.4334 |
| Individual 2 | 1.9878 | 2.6898 | 2.9882 | 0.5909 | 0.3431 |
| Individual 3 | 1.7877 | 1.9722 | 2.8972 | 0.6569 | 0.5409 |
| Average | 1.8842 | 2.476 | 2.9655 | 0.5967 | 0.4391 |
| Standard | ±0.10023524 | ±0.43799708 | ±0.06028543 | ±0.05756703 | ±0.09902456 |
| deviation | | | | | |

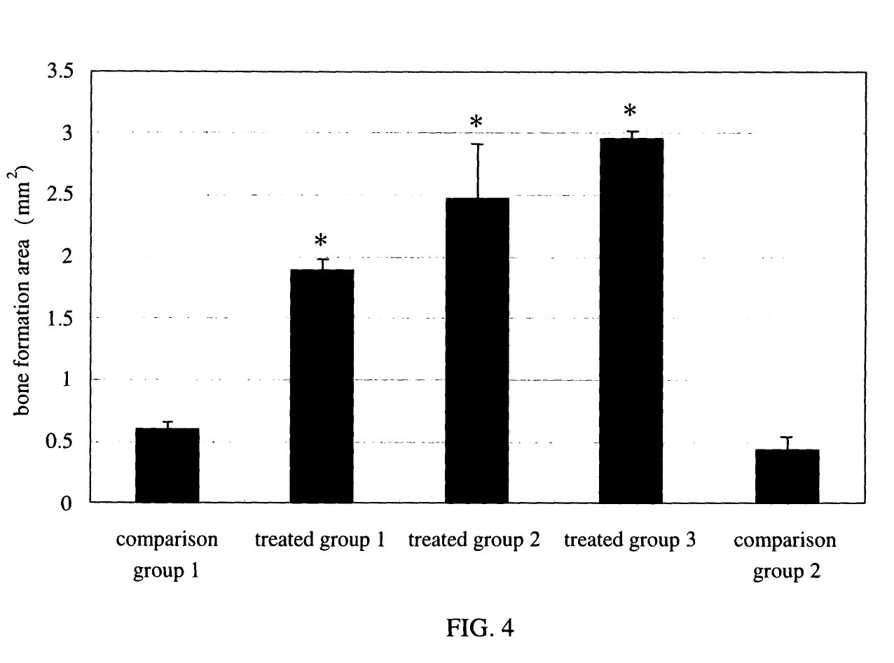

In FIG. 4, the above results of measuring the area of the regenerated alveolar bone are shown in a graph. In each group that had been treated with the respective polyphosphate-collagen complexes, the effects of promoting the regeneration of alveolar bone were significantly higher with P<0.01 (symbol “*”), compared with the comparison group 1 that had been treated only with collagen.

The above results indicate that in the polyphosphate-collagen complex-treated groups, the longer the average chain length of polyphosphates composing the polyphosphate-collagen complex to be injected, the larger the area of the regenerated alveolar bone. Thus, it was demonstrated that the polyphosphate-collagen complex of the present invention containing polyphosphates having a longer average chain length had a higher effect on the promotion of tissue regeneration.

With the polyphosphate-collagen complex of the present invention, a material that is useful as a medical material and is capable of effectively exerting the action to promote tissue regeneration can be provided.

The invention is not to be limited in scope by the specific embodiments illustrated in the specification, and functionally equivalent methods and components are within the scope of the invention. Indeed various modifications of the invention, in addition to those shown and described herein will become apparent to those skilled in the art from foregoing description. Such modifications are intended to fall within the scope of the appended claims.

All references cited herein, including patent applications, patents and other publications, are incorporated by reference herein in their entireties for all purposes.

What is claimed is:

1. A polyphosphate-collagen complex, comprising at least one polyphosphate bound to collagen.

2. The complex according to claim 1, wherein the polyphosphate is at least one type as represented by a general formula: (PₙO₃ₙ₊₁)⁽ⁿ⁺²⁾⁻ (wherein “n” indicates an integer between 2 and 5000).

3. The complex according to claim 1, wherein the polyphosphate is a linear condensed polyphosphate.

4. The complex according to claim 2, wherein “n” in the formula is an integer between 20 and 2000.

5. The complex according to claim 1, wherein the collagen is an atelocollagen.

6. The complex according to claim 1, wherein the weight ratio of polyphosphate to collagen is between 0.1% :99.9% and 20% :80%.

7. A medical material for promoting tissue regeneration, containing the complex of claim 1.

8. A medical material for treating periodontal disease, containing the complex of claim 1.

9. A process for preparing a polyphosphate-collagen complex, which comprises mixing a polyphosphate solution at a concentration of 0.5 to 10% by weight with a collagen solution at a concentration of 0.1 to 10% by weight, and collecting the generated precipitate.

10. The process according to claim 9, wherein the mixing is performed under pH conditions of between 5.0 and 8.0.

11. The process according to claim 9, wherein the polyphosphate solution is a solution of at least one type of polyphosphoric acids that is represented by a general formula: (PₙO₃ₙ₊₁₎ⁿ⁺²⁾ H (wherein “n” indicates an integer between 2 and 5000) or a salt thereof.

12. The process according to claim 11, wherein “n” in the formula is an integer between 20 and 2000.

13. The process according to claim 9, wherein the polyphosphate solution is a solution of polyphosphoric acids having an average chain length of 60 to 70 or a salt thereof.

14. The process according to claim 9, wherein the collagen contained in the collagen solution is a water-soluble collagen.

15. The process according to claim 14, wherein the collagen contained in the collagen solution is an atelocollagen.

16. The process according to claim 9, wherein the weight ratio of polyphosphate to collagen in the polyphosphate-collagen complex is between 0.1% :99.9% and 20% :80%.

17. A method for concentrating and separating polyphosphates having an average chain length of 60 to 70, which comprises mixing, at a volume ratio of between 2:1 and 9:1, a hexametaphosphate solution at a concentration of 0.1 to 10% by weight with 87 to 100% ethanol, and separating the precipitated polyphosphates from a reaction solution.

18. A method for promoting tissue regeneration, which comprises applying the complex of claim 1 to the tissue, for which tissue regeneration should be promoted.

19. The method according to claim 18, wherein a subject tissue is periodontal tissue, bone, or burned or wounded tissue.
